(19) United States
(12) Patent Application Publication
(10) Pub. No.: US 2023/0069381 A1
(43) Pub. Date: Mar. 2, 2023

(54) SLEEPINESS PREDICTION SYSTEM AND SLEEPINESS PREDICTION METHOD

(71) Applicant: Panasonic Intellectual Property Management Co., Ltd., Osaka (JP)

(72) Inventors: Yu NAKASHIMA, Kanagawa (JP); Tetsuo MATSUSE, Osaka (JP); Momoha TAKAHASHI, Kanagawa (JP)

(73) Assignee: Panasonic Intellectual Property Management Co., Ltd., Osaka (JP)

(21) Appl. No.: 17/881,282

(22) Filed: Aug. 4, 2022

(30) Foreign Application Priority Data
Aug. 31, 2021 (JP) 2021-141314
Apr. 14, 2022 (JP) 2022-066876

(51) Int. Cl. B60W 40/08; A61B 5/00; A61B 5/18; A61M 21/00; B60W 30/18; B60W 50/14; G16H 10/20

(52) U.S. Cl. CPC B60W 40/08; A61B 5/6893; A61B 5/18; A61B 5/7275; A61M 21/00; B60W 30/18009; B60W 50/14; G16H 10/20; A61B 5/0077; A61M 2021/0083; B60W 2540/229; B60W 2420/42

(57) ABSTRACT

A sleepiness prediction system includes a lifelog obtainer, a route information obtainer, and a sleepiness predictor. The lifelog obtainer obtains a lifelog including at least a get-up time of an occupant of a vehicle and a boarding time at which the occupant boards the vehicle. The route information obtainer obtains route information regarding a route to a destination of the vehicle. The sleepiness predictor predicts a sleepiness level of the occupant while the occupant is in the vehicle, based on the lifelog and the route information.

### CROSS REFERENCE TO RELATED APPLICATIONS

The present application is based on and claims priority of Japanese Patent Application No. 2021-141314 filed on Aug. 31, 2021, and priority of Japanese Patent Application No. 2022-066876 filed on Apr. 14, 2022.

### FIELD

The present disclosure relates to a sleepiness prediction system and a sleepiness prediction method for predicting sleepiness of occupants of a vehicle.

### BACKGROUND

Patent Literatures (PTLs) 1 and 2 each disclose a sleepiness prediction device. For example, the sleepiness prediction device disclosed in PTL 1 includes: a database which stores the types of roads and parameters relating to predicted values of sleepiness levels of a driver of a vehicle in association with each other; and a road type obtainer which obtains the type of each of roads in a driving route when the driving route has been set for the vehicle. In addition, the sleepiness prediction device predicts a driving distance of the vehicle along the driving route which increases the sleepiness level of the driver by obtaining, from the database, parameters corresponding to the types of the respective roads obtained by the road type obtainer.

In addition, PTL 3 discloses an alert control device. The alert control device includes an estimator which estimates a point of time at which sleepiness occurred, based on a biological rhythm stored in a storage. The alert control device further includes an alert controller which executes control for (i) causing an alert to be output to a driver, (ii) lowering the implementation standards for outputting an alert, or (iii) increasing the degree of outputting an alert, at the point of time estimated by the estimator or before the point of time.

### CITATION LIST

### Patent Literature

PTL 1: Japanese Patent No. 5696632
PTL 2: Japanese Patent No. 5691967
PTL 3: Japanese Unexamined Patent Application Publication No. 2016-133850

### SUMMARY

However, the devices according to PTLs 1 to 3 have room for improvement.

In view of this, the present disclosure provides a sleepiness prediction system, etc., which enable further improvement.

A sleepiness prediction system according to an aspect of the present disclosure includes a lifelog obtainer, a route information obtainer, and a sleepiness predictor. The lifelog obtainer obtains a lifelog including at least a get-up time of an occupant of a vehicle and a boarding time at which the occupant boards the vehicle. The route information obtainer obtains route information regarding a route to a destination of the vehicle. The sleepiness predictor which predicts a sleepiness level of the occupant while the occupant is in the vehicle, based on the lifelog and the route information.

A sleepiness prediction method according to an aspect of the present disclosure includes: obtaining a lifelog including at least a get-up time of an occupant of a vehicle and a boarding time at which the occupant boards the vehicle; obtaining route information regarding a route to a destination of the vehicle; and predicting a sleepiness level of the occupant while the occupant is in the vehicle, based on the lifelog and the route information.

The sleepiness prediction method, etc., of the present disclosure enable further improvement.

### BRIEF DESCRIPTION OF DRAWINGS

These and other advantages and features of the present disclosure will become apparent from the following description thereof taken in conjunction with the accompanying drawings that illustrate a specific embodiment of the present disclosure,

FIG. 1 is a block diagram illustrating an outline of a sleepiness prediction system according to Embodiment 1.

FIG. 2 is a diagram explaining various kinds of parameters that are used by the sleepiness prediction system according to Embodiment 1,

FIG. 3 is a diagram indicating an outline of one example of a difference correction process that is performed by a sleepiness predictor of the sleepiness prediction system according to Embodiment 1.

FIG. 4 is a diagram indicating an outline of another example of a difference correction process that is performed by the sleepiness predictor of the sleepiness prediction system according to Embodiment 1.

FIG. 5 is a diagram illustrating an outline of an example of a notification that is provided by a notifier of the sleepiness prediction system according to Embodiment 1.

FIG. 6 is a diagram explaining physical condition icons that are displayed on a display according to Embodiment 1.

FIG. 7 is a flow chart indicating an example of an operation that is performed by the sleepiness prediction system according to Embodiment 1.

FIG. 8 is a block diagram illustrating an outline of a sleepiness prediction system according to Embodiment 2.

FIG. 9 is a flow chart indicating an example of an operation that is performed by the sleepiness prediction system according to Embodiment 2.

### DESCRIPTION OF EMBODIMENTS

The present disclosure has an object to provide a sleepiness prediction system, etc., which make it possible to increase accuracy in prediction of sleepiness (a sleepiness level) of each of occupants of a vehicle.

A sleepiness prediction system according to an aspect of the present disclosure includes a lifelog obtainer, a route information obtainer, and a sleepiness predictor. The lifelog obtainer obtains a lifelog including at least a get-up time of an occupant of a vehicle and a boarding time at which the occupant boards the vehicle. The route information obtainer obtains route information regarding a route to a destination of the vehicle. The sleepiness predictor predicts a sleepiness level of the occupant while the occupant is in the vehicle, based on the lifelog and the route information.

With this, the sleepiness level of the occupant is predicted based on the route to the destination of the vehicle which can affect the sleepiness of the occupant and the get-up time and the boarding time of the occupant. This provides an advantageous effect of being able to increase accuracy in prediction of the sleepiness of the occupant of the vehicle.

In particular, since the sleepiness level of the occupant is predicted based on the get-up time and the boarding time of the occupant, in other words, in consideration of an active period from when the occupant gets up to when the occupant boards the vehicle, it is expected that the accuracy in prediction of the sleepiness of the occupant further increases compared with the case in which the sleepiness level of the occupant is predicted without considering the active period. For example, even when the sleepiness level at the time when the occupant boards the vehicle is the same, the sleepiness level may transit differently depending on the length of the active period before the occupant boards the vehicle.

The sleepiness prediction system according to another aspect of the present disclosure calculates an active period coefficient corresponding to an active period which is a difference between the get-up time and the boarding time obtained by the lifelog obtainer. In addition, the sleepiness predictor calculates a predicted driving period coefficient corresponding to a predicted driving period required to drive the route, based on the route information obtained by the route information obtainer. In addition, the sleepiness predictor calculates a route type coefficient corresponding to a type of the route, based on the route information obtained by the route information obtainer. The sleepiness predictor then predicts the sleepiness level of the occupant while the occupant is in the vehicle, based on the active period coefficient, the predicted driving period coefficient, and the route type coefficient.

This provides an advantageous effect of being able to further increase the accuracy in prediction of the sleepiness level of the occupant while the occupant is in the vehicle in consideration of the active period of the occupant, the predicted driving period of the vehicle, and the type of the route.

In the sleepiness prediction system according to another aspect of the present disclosure, the lifelog obtainer further obtains a go-to-bed time of the occupant as a part of the lifelog.

This provides an advantageous effect of being able to further increase the accuracy in prediction of the sleepiness level of the occupant while the occupant is in the vehicle in consideration of the go-to-bed time of the occupant.

In the sleepiness prediction system according to another aspect of the present disclosure, the sleepiness predictor calculates a sleep period coefficient corresponding to a sleep period which is a difference between the go-to-bed time and the get-up time obtained by the lifelog obtainer. The sleepiness predictor then predicts the sleepiness level of the occupant while the occupant is in the vehicle, further based on the sleep period coefficient.

This provides an advantageous effect of being able to further increase the accuracy in prediction of the sleepiness level of the occupant while the occupant is in the vehicle in consideration of the sleep period of the occupant.

The sleepiness prediction system according to another aspect of the present disclosure further includes a state information obtainer which obtains state information regarding a state of the occupant at a time of boarding the vehicle. The sleepiness predictor calculates a current sleepiness level of the occupant based on the state information, and predicts the sleepiness level of the occupant while the occupant is in the vehicle, further based on the current sleepiness level.

This provides an advantageous effect of being able to further increase the accuracy in prediction of the sleepiness level of the occupant while the occupant is in the vehicle in consideration of the current sleepiness level of the occupant.

In the sleepiness prediction system according to another aspect of the present disclosure, the sleepiness predictor re-calculates a current sleepiness level of the occupant every time a predetermined time has elapsed from the boarding time or an immediately previous predetermined time. When a difference is made between the current sleepiness level re-calculated and the sleepiness level of the occupant while the occupant is in the vehicle, the sleepiness predictor then corrects the sleepiness level of the occupant while the occupant is in the vehicle so as to eliminate the difference.

This provides an advantageous effect of being able to further increase the accuracy in prediction of the sleepiness level of the occupant while the occupant is in the vehicle by correcting the sleepiness level of the occupant while the occupant is in the vehicle every time a predetermined time has elapsed.

The sleepiness prediction system according to another aspect of the present disclosure further includes a notifier which notifies the occupant of the sleepiness level of the occupant while the occupant is in the vehicle, the sleepiness level of the occupant being predicted by the sleepiness predictor.

This allows the occupant to know the transition in future sleepiness level, which provides an advantageous effect that it is easy for the occupant to take an action for increasing safety in driving the vehicle, for example, by taking a rest.

In the sleepiness prediction system according to another aspect of the present disclosure, the lifelog obtainer further obtains a go-to-bed time of the occupant as a part of the lifelog. The notifier further notifies the occupant of physical condition information of the occupant based on an active period and a sleep period, the active period being a difference between the get-up time and the boarding time obtained by the lifelog obtainer, the sleep period being a difference between the go-to-bed time and the get-up time obtained by the lifelog obtainer.

This allows the occupant to know his/her physical condition at the time when the occupant boards the vehicle, which provides an advantageous effect that it is easy for the occupant to know the correlation between the transition in future sleepiness level and his/her physical condition.

In the sleepiness prediction system according to another aspect of the present disclosure, the state information obtainer obtains, as the state information, an image of the occupant from a camera which is mounted on the vehicle and captures an image of the occupant.

With this, it is possible to obtain images of parts having correlation with the sleepiness of the occupant. Examples of the parts include eyelids of the occupant. This provides an advantageous effect that it becomes easy to obtain the current sleepiness level of the occupant.

In the sleepiness prediction system according to another aspect of the present disclosure, the lifelog obtainer obtains at least a part of the lifelog from a wearable device worn by the occupant.

With this, the occupant wears the wearable device daily, which provides an advantageous effect that it is easy to increase accuracy in obtainment of the lifelog.

In the sleepiness prediction system according to another aspect of the present disclosure, the route information obtainer obtains the route information from a navigation system mounted on the vehicle.

This provides an advantageous effect that it is easy to obtain the route information compared with the case in which the route information is obtained from the navigation system mounted on an information terminal such as a smartphone.

In the sleepiness prediction system according to another aspect of the present disclosure, the occupant comprises a driver and one or more passengers, and the sleepiness predictor predicts each of a sleepiness level of the driver and a sleepiness level of each of the one or more passengers, and notifies, via the notifier, each of the driver and the one or more passengers of the sleepiness level of the driver and the sleepiness level of the passenger.

This provides an advantageous effect that also the passenger can know whether the driver will be able to be awake until the vehicle arrives at the destination. This also provides an advantageous effect that the passenger can know the point of time at which switching of drivers will be necessary when it is predicted that the driver will not be able to be awake.

The sleepiness prediction system according to another aspect of the present disclosure further includes a waking stimulator which gives a waking stimulus to the occupant. When (i) the sleepiness predictor determines that a future sleepiness level of the driver will reach a maximum predetermined value and (ii) the current sleepiness level of the driver reaches a first predetermined value, the sleepiness predictor outputs, to the waking stimulator, a driver waking stimulus signal which instructs giving of a waking stimulus to the driver, and notifies, via the notifier, at least one of the one or more passengers that the waking stimulus is being given to the driver.

This provides advantageous effects of being able to wake the driver at a stage of a level (first predetermined value) at which the driver feels sleepy, and to notify in advance the passenger of the possibility of taking over of the driving.

In the sleepiness prediction system according to another aspect of the present disclosure, when the sleepiness predictor determines that the future sleepiness level of the driver will reach a second predetermined value higher than the first predetermined value, the sleepiness predictor outputs, to the waking stimulator, a passenger waking stimulus signal which instructs giving of a waking stimulus to the at least one of the one or more passengers.

This provides an advantageous effect of being able to give a waking stimulus to the passenger to provide the passenger with pre-notification regarding the taking over of the driving.

In the sleepiness prediction system according to another aspect of the present disclosure, when the sleepiness predictor determines that (i) a future sleepiness level of the driver will reach a maximum predetermined value and (ii) the current sleepiness level of the driver exceeds a second predetermined value lower than the maximum predetermined value, the sleepiness predictor notifies, via the notifier, the driver and a passenger having a lower sleepiness level than the current sleepiness level of the driver among the one or more passengers of switching drivers from the driver to the passenger having the lower sleepiness level.

With this, switching to the passenger having a sleepiness level lower than that of the driver is urged, which provides an advantageous effect of being able to continue safety driving to the destination.

In the sleepiness prediction system according to another aspect of the present disclosure, when the one or more passengers comprise a plurality of passengers, the sleepiness predictor notifies, via the notifier, at least a passenger having a lowest sleepiness level among the plurality of passengers of switching drivers from the driver to the passenger having the lowest sleepiness level.

This provides an advantageous effect of increasing the likelihood of safety driving to the destination by means of the passenger having the lowest sleepiness taking over the driving among all the occupants.

The sleepiness prediction system according to another aspect of the present disclosure further includes an automatic stop controller which executes control for causing the vehicle to automatically stop. When the sleepiness predictor notifies, via the notifier, the driver and the passenger of switching of drivers from the driver to the passenger, the sleepiness predictor outputs, to the automatic stop controller, an automatic stop signal for causing the vehicle to automatically stop at a road shoulder.

This provides an advantageous effect of enabling safety stop of the vehicle and switching to the passenger.

In the sleepiness prediction system according to another aspect of the present disclosure, the lifelog obtainer further obtains, as a part of the lifelog, driver's license holder information of a passenger who holds a driver's license among the one or more passengers, and the sleepiness predictor notifies, via the notifier, at least the passenger who holds the driver's license of switching of drivers from the driver to the passenger who holds the driver's license.

This provides an advantageous effect of enabling switching to the more appropriate passenger.

The sleepiness prediction system according to another aspect of the present disclosure further includes a notifier which notifies the occupant of the sleepiness level of the occupant while the occupant is in the vehicle, the sleepiness level of the occupant being predicted by the sleepiness predictor. In the sleepiness prediction system, the occupant comprises a driver and one or more passengers, the sleepiness predictor: predicts each of a sleepiness level of the driver and a sleepiness level of each of the one or more passengers; predicts the sleepiness level of the passenger using a coefficient in a case where the route is simplest as the route type coefficient, when predicting the sleepiness level of the passenger; and notifies, via the notifier, each of the driver and the passenger of the predicted sleepiness level of the driver and the predicted sleepiness level of the passenger.

This provides an advantageous effect that also the passenger can know whether the driver will be able to be awake until the vehicle arrives at the destination. This also provides an advantageous effect that the passenger can know the point of time at which switching of drivers will be necessary when it is predicted that the driver will not be able to be awake. Furthermore, since the sleepiness level of the passenger is predicted using the route type coefficient in the case where the route is simplest, an advantageous effect of being able to accurately predict the sleepiness level of the passenger who does not drive the vehicle more is provided.

A sleepiness prediction method according to an aspect of the present disclosure includes: obtaining a lifelog including at least a get-up time of an occupant of a vehicle and a boarding time at which the occupant boards the vehicle; obtaining route information regarding a route to a destination of the vehicle; and predicting a sleepiness level of the occupant while the occupant is in the vehicle, based on the lifelog and the route information.

With this, the sleepiness level of the occupant is predicted based on the route to the destination of the vehicle which can affect the sleepiness of the occupant and the get-up time and the boarding time of the occupant. This provides an advantageous effect of being able to increase accuracy in prediction of the sleepiness of the occupant of the vehicle.

In particular, since the sleepiness level of the occupant is predicted based on the get-up time and the boarding time of the occupant, in other words, in consideration of an active period from when the occupant gets up to when the occupant boards the vehicle, it is expected that the accuracy in prediction of the sleepiness of the occupant further increases compared with the case in which the sleepiness level of the occupant is predicted without considering the active period. For example, even when the sleepiness level at the time when the occupant boarded the vehicle is the same, the sleepiness level may transit differently depending on the length of the active period before the occupant boards the vehicle.

Hereinafter, Embodiments 1 and 2 are described specifically with reference to the drawings.

It is to be noted that each of Embodiments 1 and 2 to be described below indicates a general or a specific example. The numerical values, the shapes, the materials, the constituent elements, the arrangement and connection of the constituent elements, the steps, the order of the steps, etc., indicated in the following Embodiments 1 and 2 are mere examples, and therefore do not intend to limit the present disclosure. In addition, the constituent elements not recited in any of the independent claims among the constituent elements of Embodiments 1 and 2 are described as optional constituent elements.

In addition, each of the drawings is a schematic diagram, and thus is not always illustrated precisely. Throughout the drawings, the same constituent elements are assigned with the same reference signs.

### Embodiment 1

### [A Configuration]

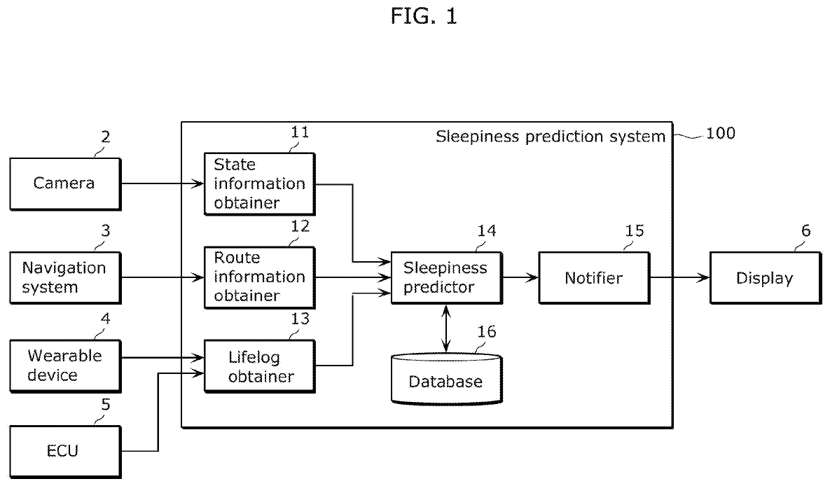

FIG. 1 is a block diagram illustrating an outline of sleepiness prediction system 100 according to Embodiment 1. Sleepiness prediction system 100 according to Embodiment 1 is a system which is used for, for example, a moving body such as a vehicle and which is for supporting driving of the vehicle. Sleepiness prediction system 100 is implemented as an in-vehicle device in Embodiment 1, but sleepiness prediction system 100 may be implemented as an external device which is brought from outside the vehicle. The occupant who is a target of sleepiness prediction system 100 is a driver who is seated on a driver's sheet in Embodiment 1, but a target may be an occupant who is seated on an assistant driver's sheet or a rear sheet other than the driver.

As illustrated in FIG. 1, sleepiness prediction system 100 includes state information obtainer 11, route information obtainer 12, lifelog obtainer 13, sleepiness predictor 14, notifier 15, and database 16.

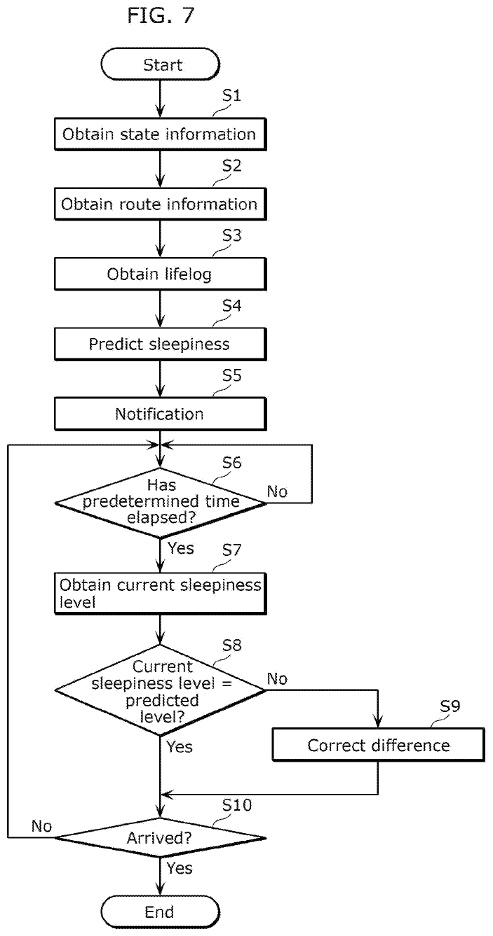

State information obtainer 11 obtains state information regarding a state of an occupant at the time when the occupant boards the vehicle. State information obtainer 11 is a subject which performs Step S1 (see FIG. 7) included in a sleepiness prediction method. The state information is information regarding a state of a body part of the occupant who is seated on a sheet of the vehicle. Examples of the state include swing of the head, the positions of the eyelids, the posture, etc., of the occupant. The state information may include information which is likely to present a tendency according to the sleepiness of the occupant. In Embodiment 1, the state information includes information regarding the positions of the eyelids of the occupant.

In Embodiment 1, state information obtainer 11 obtains an image of the occupant captured by camera 2 mounted on the vehicle as the state information from camera 2. Camera 2 captures the image in which the eyelids of the occupant seated on the sheet are present by capturing the image at a position in front of the occupant or at a room mirror position. State information obtainer 11 obtains the image captured by camera 2 by performing wired communication or wireless communication with camera 2.

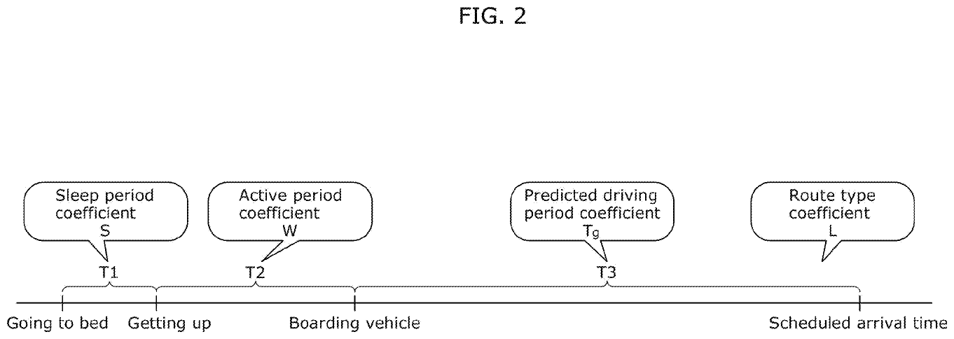

Route information obtainer 12 obtains route information regarding a route to a destination of the vehicle. Route information obtainer 12 is a subject which performs Step S2 (see FIG. 7) included in the sleepiness prediction method. The state information may include information regarding the type of the route in addition to predicted driving period T3 (see FIG. 2) required to drive the route from a current location of the vehicle to the destination. Specifically, the route information may include, as the information regarding the type of the route, for example, information indicating whether the route is a highway, a road exclusive for automobiles, or another road such as a local road, information indicating whether the route is straight or curved, and/or information indicating whether the route is flat, an uphill, or a downhill. The route information may include, as the information regarding the type of the route, for example, information indicating whether the route includes a tunnel, information indicating whether the route includes a junction, and/or information indicating whether the number of lanes of the route is 3 or more.

In Embodiment 1, route information obtainer 12 obtains route information from navigation system 3 mounted on the vehicle. Navigation system 3 is a car navigation system. When receiving, from the occupant, an input of an operation for setting a destination, navigation system 3 sets a route from the current location of the vehicle to the destination, and calculates a scheduled arrival time required to arrive at the destination (that is, calculates predicted driving period T3). Route information obtainer 12 obtains information including the route which has been set by navigation system 3 and predicted driving period T3 that is the route information by performing wired communication or wireless communication with navigation system 3.

Lifelog obtainer 13 obtains a lifelog including a get-up time of the occupant of the vehicle and a boarding time at which the occupant boards the vehicle. Lifelog obtainer 13 is a subject which performs Step S3 (see FIG. 7) included in the sleepiness prediction method. The lifelog is information regarding a daily life of the occupant. In Embodiment 1, lifelog obtainer 13 further obtains a go-to-bed time of the occupant as a part of the lifelog in addition to the get-up time and the boarding time. The get-up time is a time at which the occupant got up in the day on which the occupant was scheduled to board the vehicle or a time at which the occupant got up in the previous day. The boarding time is the time at which the occupant boarded the vehicle in the day on which the occupant was scheduled to board the vehicle. The go-to-bed time is a time at which the occupant went to bed in the day on which the occupant was scheduled to board the vehicle or a time at which the occupant went to bed in the previous day.

In Embodiment 1, lifelog obtainer 13 obtains at least a part of the lifelog from wearable device 4 worn by the occupant, and obtains at least a part of the lifelog from electronic control unit (ECU) 5 mounted on the vehicle.

Wearable device 4 is a device that is for example a smart watch or smart glasses which can be put on a wrist, an arm, the head, or another part of the occupant. Wearable device 4 includes a function of measuring numerical data regarding an activity of the occupant. Examples of the numerical data regarding the activity includes the number of steps, a blood pressure, a heart rate, or other numerical data. Lifelog obtainer 13 obtains a get-up time and a go-to-bed time of the occupant measured by wearable device 4, that is a part of the lifelog, by performing wireless communication with wearable device 4.

ECU 5 measures, for example, the tarn e at which the power of the vehicle is turned on after the occupant boarded the vehicle as the boarding time. It is to be noted that ECU 5 may measure, for example, the time at which an input of an operation for setting the destination by the occupant was received by navigation system 3 as the boarding time. Lifelog obtainer 13 obtains the information including the boarding time measured by ECU 5, that is a part of the lifelog, by performing wired communication or wireless communication with ECU 5.

Sleepiness predictor 14 predicts the sleepiness level of the occupant while the occupant is in the vehicle, based on the lifelog obtained by lifelog obtainer 13 and the route information obtained by route information obtainer 12. Sleepiness predictor 14 is a subject which performs Steps S4 and Steps S6 to S10 (see FIG. 7) included in the sleepiness prediction method. The sleepiness level of the occupant while the occupant is in the vehicle is predicted by sleepiness predictor 14 as described above, and the sleepiness level of the occupant while the occupant is in the vehicle is hereinafter also simply referred to as “a predicted level”.

In Embodiment 1, as indicated in FIG. 2, sleepiness predictor 14 predicts a sleepiness level (predicted level) of the occupant while the occupant is in the vehicle, based on activity period coefficient W, predicted driving period coefficient Tg, and route type coefficient L. In addition, in Embodiment 1, as indicated in FIG. 2, sleepiness predictor 14 predicts a sleepiness level (predicted level) of the occupant while the occupant is in the vehicle, further based on sleep period coefficient S.

FIG. 2 is a diagram explaining various kinds of parameters that are used by sleepiness prediction system 100 according to Embodiment 1. As indicated in FIG. 2, sleep period coefficient S is a parameter corresponding to sleep period T1 which is the difference between the go-to-bed time and the get-up time obtained by lifelog obtainer 13. Sleep period coefficient S varies, for example, in a numerical range from 0 to 5, and is greater as sleep period T1 is shorter and is smaller as sleep period T1 is longer. In Embodiment 1, sleepiness predictor 14 calculates sleep period coefficient S by referring to data which defines a correlation between sleep period T1 and sleep period coefficient S stored in database 16. For example, sleepiness predictor 14 calculates sleep period coefficients S as follows: “5” when sleep period T1 is less than 1 hour; “3” when sleep period T1 is greater than or equal to 5 hours and less than 7 hours; and “0” when sleep period T1 is greater than or equal to 10 hours.

As indicated in FIG. 2, active period coefficient W is a parameter corresponding to active time T2 which is the difference between the get-up time and the boarding time obtained by lifelog obtainer 13. Active period coefficient W varies, for example, in a numerical range from 0 to 5, and is greater as active period T2 is longer and is smaller as active period 12 is shorter. In Embodiment 1, sleepiness predictor 14 calculates active period coefficient W by referring to data which defines a correlation between active period T2 and active period coefficient W stored in database 16, For example, sleepiness predictor 14 calculates active period coefficients W as follows: “0” when active period T2 is less than 4 hours; “3” when active period T2 is greater than or equal to 12 hours and less than 16 hours; and “5” when active period T2 is greater than or equal to 20 hours.

As indicated in FIG. 2, predicted driving period coefficient Tg is a parameter corresponding to predicted driving period T3, based on the route information obtained by route information obtainer 12. Predicted driving period coefficient Tg varies, for example, in a numerical range from 0.1 to 1, and is greater as predicted driving period T3 is longer and is smaller as predicted driving period T3 is shorter. In Embodiment 1, sleepiness predictor 14 calculates predicted driving period coefficient Tg by referring to data which defines a correlation between predicted driving period T3 and predicted driving period coefficient Tg stored in database 16, For example, sleepiness predictor 14 calculates predicted driving period coefficient Tg as “0” when predicted driving period T3 is 0 hour, and calculates, every time predicted driving period T3 becomes longer by 10 minutes, predicted driving period coefficient Tg incremented by “0.05”. Sleepiness predictor 14 finally calculates predicted driving period coefficient Tg as “1” when, for example, predicted driving period T3 is 200 minutes or longer.

Although predicted driving period T3 and predicted driving period coefficient Tg has a linear correlation in Embodiment 1, it is to be noted that driving period T3 and predicted driving period coefficient Tg may have a non-linear correlation in which, for example, predicted driving period coefficient Tg increases abruptly as predicted driving period T3 increases.

As indicated in FIG. 2, route coefficient L is a parameter corresponding to the type of the route on which the vehicle drives in predicted driving period T3, based on the route information obtained by route information obtainer 12. Route type coefficient L varies, for example, in a numerical range from 0 to 5, and is greater as the route type is simpler and is smaller as the route type is more complicated. That “the route type is complicated” here means that, for example, the vehicle needs to be driven differently comparatively often by, for example, increasing the speed, decreasing the speed, being stopped, changing lanes due to traffic signals or presence of walkers. Sleepiness predictor 14 calculates route type coefficient L, for example, by referring to data which defines a correlation between the route type and route type coefficient L stored in database 16. For example, sleepiness predictor 14 calculates route type coefficient L to be “5” when the route is a highway, is straight and flat, includes a tunnel, and the number of lanes is 3 or more. In another example, sleepiness predictor 14 calculates route type coefficient L to be “2.5” when the route is a highway, is curved and an uphill or a downhill, includes a junction, and the number of lanes is 3 or more. In another example, sleepiness predictor 14 calculates route type coefficient L to be “0” when the route is a local road (on which the driver is often required to increase and decrease the speed of the vehicle, stop the vehicle, and cause the vehicle to turn right or left at intersections).

Furthermore, in Embodiment 1, sleepiness predictor 14 calculates a current sleepiness level of the occupant based on state information obtained by state information obtainer 11, and predicts a sleepiness level (predicted level) of the occupant while the occupant is in the vehicle, further based on the current sleepiness level. For example, sleepiness predictor 14 executes, as necessary, an image analysis process on an image in which the eyelids of the occupant are present obtained from camera 2, and calculates a current sleepiness level of the occupant, based on a representative value (for example, an average value, or the like) of the positions of the eyelids of the occupant in a defined period. For example, the positions of the eyelids can be defined as an eye-open degree assuming that the full-open state of the eyes is 100% and the full-closed state of the eyes is 0%. In Embodiment 1, sleepiness predictor 14 calculates the current sleepiness level of the occupant by referring to data which defines correlations between the sleepiness levels and the representative levels each indicating the positions of the eyelids and the speed(s) of the motion(s) of the eyelids in the defined period stored in database 16.

In Embodiment 1, sleepiness predictor 14 calculates the current sleepiness level of the occupant at one of 5 stages of “Level 1” to “Level 5”, and likewise calculates the predicted level at one of 5 stages of “Level 1” to “Level 5”. “Level 1” is a level in the case where it is estimated that the occupant does not feel sleepy at all. Level 2 is a level in the case where it is estimated that the occupant feels slightly sleepy. Level 3 is a level in the case where it is estimated that the occupant feels sleepy. Level 4 is a level in the case where it is estimated that the occupant feels very sleepy. Level 5 is a level in the case where it is estimated that the occupant feels extremely sleepy.

As one example, the predicted level is calculated according to the following expression (1) using route type coefficient L, sleep period coefficient S, predicted driving period coefficient Tg, active period coefficient W, and current sleepiness level N, In the expression (1), “D” denotes a predicted level. Also in the expression (1), “N”, “S”, and “W” are each a constant which is determined at the time when the driver boarded the vehicle, but “D”, “L”, and “Tg” each change with time from the current point of time (here, the point of time at which the driver boards the vehicle). Accordingly, in the expression (1), “D”, “L”, and “Tg” are each represented as a function of an elapsed time “t” from the current point of time.

[Math. 1]

D(t)=Tg(t)(S+W+L(t))+N (1)

Here, although “D(t)” is calculated as a value in a numerical range from 0 to 20 in the expression (1), the predicted level is one of the 5 stages from “Level 1” to “Level 5” as described above. Accordingly, sleepiness predictor 14 calculates a predicted level to be “Level 5” in all cases where the calculated “D(t)” is 5 or more. It is to be noted that, when the calculated “D(t)” is less than 5, a numerical value with a decimal number is used as “D(t)” at the time of calculating expressions (2) to (4) to be described later.

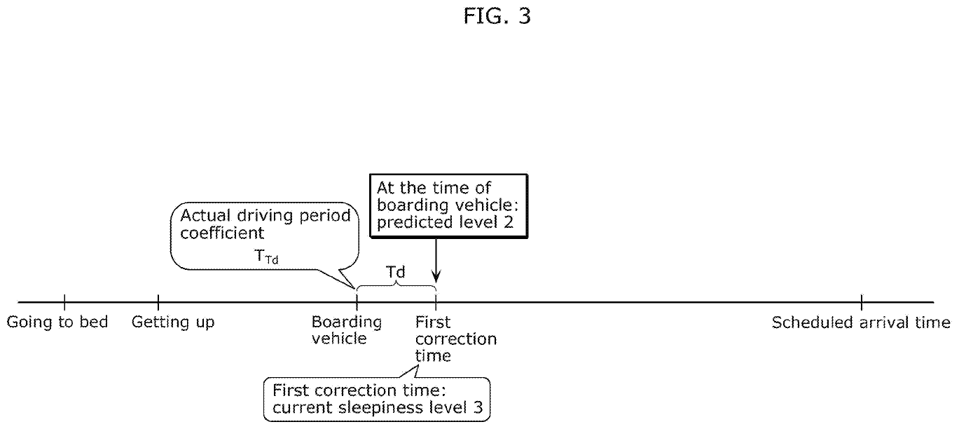

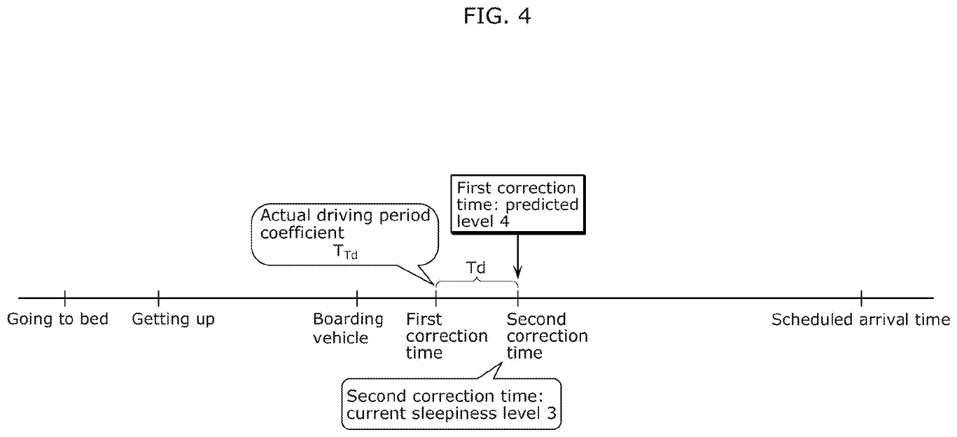

Furthermore, in Embodiment 1, sleepiness predictor 14 determines whether a difference correction process is to be executed on a predicted level every time predetermined time Td (see FIGS. 3 and 4) has elapsed from the boarding time or immediately previous predetermined time Td, in addition to the point of time at which the driver boarded the vehicle. Predetermined time Td is, for example, a minute unit, and is desirably 30 minutes.

Specifically, sleepiness predictor 14 re-calculates a current sleepiness level, based on state information obtained by state information obtainer 11 every time predetermined time Td has elapsed from the boarding time or immediately previous predetermined time Td. Sleepiness predictor 14 then compares the re-calculated current sleepiness level and the predicted sleepiness level (predicted level) while the driver is in the vehicle. When the result of the comparison indicates a match between the current sleepiness level and the predicted level, sleepiness predictor 14 does not execute any difference correction process. On the other hand, when the result of the comparison indicates a mismatch between the current sleepiness level and the predicted level, sleepiness predictor 14 executes a difference correction process.

As one example, the difference correction process is executed using the following expressions (2) to (4). In the expression (2), “D′(t)” denotes a predicted level corrected through the difference correction process, and “N′” denotes a re-calculated current sleepiness level (that is a current sleepiness level at the time when the difference correction process is executed). In addition, in the expressions (2) and (3), “Tg′(t)” denotes re-calculated predicted driving period coefficient Tg. “TTd” is a parameter corresponding to a driving period from the point of time before the current point of time by predetermined time Td to the current point of time. Hereinafter, “TTd” is referred to as “actual driving period coefficient TTd”. In addition, the expression (4) is an expression for calculating actual driving period coefficient TTd. “D” denotes a predicted level predicted by sleepiness predictor 14 at the point of time before the current point of time by predetermined time Td.

$$\begin{matrix}[ Math . 2 ] & \\ {D}^{′} ( t ) = {Tg}^{′} ( t ) ( S + W + L ( t ) ) + {N}^{′} & ( 2 )\end{matrix}$$

$${Tg}^{′} ( t ) = T g ( t ) + {T}_{Td} \qquad ( 3 )$$

$${T}_{Td} = \frac{{N}^{′} - D}{S + W + L ( t )} \qquad ( 4 )$$

Here, a specific example of the difference correction process performed by sleepiness predictor 14 is explained using FIGS. 3 and 4. FIG. 3 is a diagram indicating an outline of one example of the difference correction process performed by sleepiness predictor 14 of sleepiness prediction system 100 according to Embodiment 1. In the example indicated in FIG. 3, at a first correction time which is a point of time at which predetermined time Td has elapsed from the boarding time, the predicted level predicted by sleepiness predictor 14 at the boarding time is “Level 2” and the current sleepiness level in the first correction time is “Level 3”. In other words, the example indicated in FIG. 3 indicates a case in which the sleepiness of the occupant has progressed more than predicted. In this case, the current time is the first correction time, and the point of time before the current time by predetermined time Td corresponds to the boarding time. “N′−D” in the expression (4) is a positive number (here, 3−2=1).

FIG. 4 is a diagram indicating an outline of one example of the difference correction process performed by sleepiness predictor 14 of sleepiness prediction system 100 according to Embodiment 1. In the example indicated in FIG. 4, at a second correction time which is a point of time at which predetermined time Td has elapsed from the first correction time, the predicted level predicted by sleepiness predictor 14 at the first correction time is “Level 4” and the current sleepiness level in the second correction time is “Level 3”. In other words, the example indicated in FIG. 4 indicates a case in which the sleepiness of the occupant has not progressed more than predicted. In this case, the current time is the second correction time, and the point of time before the current time by predetermined time Td corresponds to the first correction time. “N′−D” in the expression (4) is a positive number (here, 3−4=−1).

Sleepiness predictor 14 then executes a difference correction process using the expressions (2) to (4), and corrects (updates) the predicted level. The corrected predicted level is maintained until sleepiness predictor 14 re-executes a difference correction process.

Hereinafter, a specific correction example is described, First, it is assumed that the occupant had taken normal sleep by the boarding time (t=0), and that sleep period coefficient S=2 is satisfied. In addition, it is assumed that the period from a get-up time and the boarding time is comparatively long, active period coefficient W=3 is satisfied. Next, it is assumed that the type of the route from the current location of the vehicle to a destination include many local roads, and that L(0)=0 is satisfied. In addition, it is assumed that predicted driving period T3 from the current location to the destination is 100 minutes, and that Tg(0)=0.05×(100/10)=0.5 is satisfied. Furthermore, it is assumed that the occupant is at “Level 1” indicating that the occupant does not fed sleepy when boarding the vehicle, and thus sleep level N is 1 is satisfied. When substituting these parameters to the expression (1), predicted level D(0)=0.5×(2+3+0)+1=3.5 is satisfied. Accordingly, it is predicted at the time of boarding the vehicle that the sleepiness of the occupant will be at Level 3.5 at the time when the occupant arrives at the destination, in other words, the occupant will be in a state in which the occupant feels sleepy at the time.

Next, it is assumed that 20 minutes has elapsed (t=20) after the occupant boarded the vehicle, After the 20 minutes, the remaining predicted driving period T3 is 80 minutes. Accordingly, Tg(20)=0.05×(80/10)=0.4 is satisfied. It is assumed that the route type after the 20 minutes include local roads only, L(20)=0 is satisfied. In addition, it is assumed that the occupant feels sleepy after the 20 minutes elapsed, sleepiness level N′=3 is satisfied. Here, since D in the expression (4) is the predicted level at the boarding time, D=D(0)=3.5 is satisfied. When substituting these parameters to the expression (4), TTd=(3−3.5)/(2+3+0)=−0.1 is satisfied. Accordingly, according to the expression (3), Tg′(20) is obtained from Tg′(20)=0.4−0.1=0.3. Accordingly, according to the expression (2), corrected predicted level D′(20)=0.3×(2+3+0)+3=4.5 is satisfied. Here, since the predicted level D(0) at the boarding time is 3.5, the predicted level is corrected to 4.5 after 20 minutes' driving, it is predicted that the occupant will be in a state in which the occupant feels very sleepy approximately when the occupant arrives at the destination.

Notifier 15 notifies the occupant of the sleepiness level (predicted level) of the occupant while the occupant is in the vehicle (the sleepiness level of the occupant has been predicted by sleepiness predictor 14 as described above). Notifier 15 is a subject which performs Step S5 (see FIG. 7) included in the sleepiness prediction method. In Embodiment 1, notifier 15 visually notifies the occupant of the predicted level by displaying information including the predicted level onto display 6. Display 6 is, for example, a display included in navigation system 3.

FIG. 5 is a diagram indicating an outline of one example of the notification provided by notifier 15 of sleepiness prediction system 100 according to Embodiment 1. FIG. 5 illustrates a display screen of display 6. As illustrated in FIG. 5, display 6 displays graph 61 having a vertical axis indicating sleepiness levels (predicted levels) and a horizontal level indicating elapsed time t. In graph 61, as indicated in the expressions (1) and (2), each sleepiness level (predicted level) is a function of elapsed time t. Thus, for example, each of lines A1 and A2 can represent temporal transition in sleepiness level (that is, predicted level) of the driver from a current time (for example, at the time when the driver boarded the vehicle: elapsed time t0) while the driver is in the vehicle. The transition example indicated by line A1 shows that the predicted level linearly increases with elapse of time because the type of the route is monotonous until elapsed time t1 at which the vehicle drives on a highway, and that the predicted level then gradually increases in a state in which increase rates become small because the type of the route becomes complicated compared to the type of the highway in a period during which the vehicle drives on local roads. Furthermore, the transition example indicated by line A2 shows that the entire route to the destination includes local roads only, and thus that the predicted level non-linearly increases with elapse of time, and that the maximum predicted level (sleepiness level) is reached at scheduled arrival time t2 at the destination. It is to be noted that since “Level 5” is calculated in all cases where the predicted level “5” or more as described above, the predicted level at and after scheduled arrival time t2 is a constant value of “5”. In addition, in the example indicated in FIG. 5, although both lines A1 and A2 are represented in graph 61, only one of lines A1 and A2 is actually represented in graph 61. In addition, lines A1 and A2 merely represent examples of transition in predicted level, and thus the predicted level may transit differently from transition indicated by lines A1 and A2 according to the results of prediction by sleepiness predictor 14.

In Embodiment 1, display 6 further display physical condition information. The physical condition information includes time icons 62 respectively indicating sleep time T1 and active time 12 and physical condition icons 63. In other words, notifier 15 further notifies the occupant of physical condition information about the occupant based on active period T2 and sleep period T1. Here, the physical condition information can be said as information indirectly indicating physical conditions which are of the occupant at the time when the occupant boarded the vehicle and which may affect future sleepiness of the occupant.

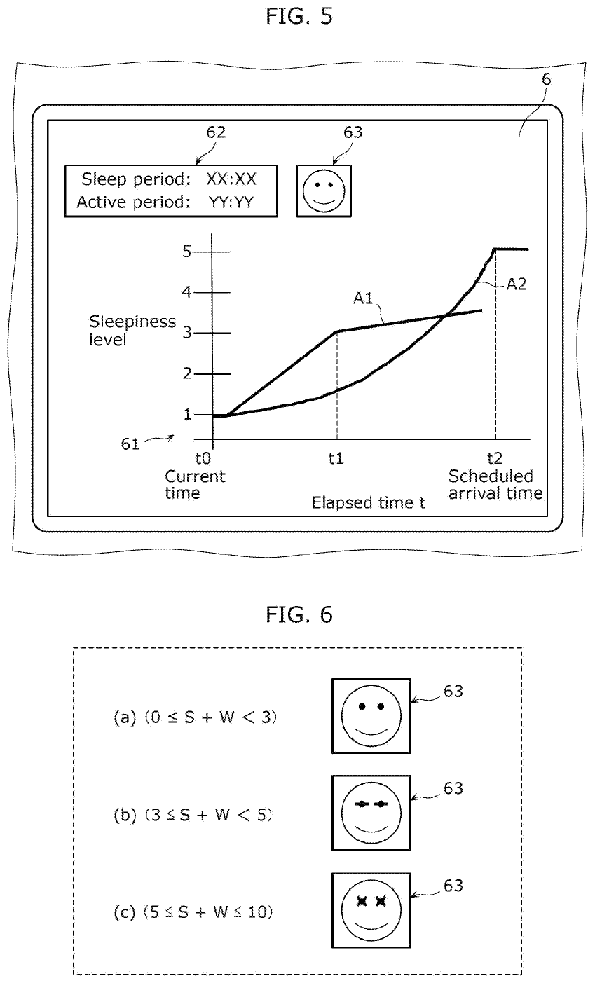

Physical condition icons 63 are represented as, for example, pictograms of human faces. As illustrated in FIG. 6, the display modes each vary depending on sleep period coefficient S corresponding to sleep time T1 and active period coefficient W corresponding to active period T2. FIG. 6 is a diagram explaining physical icons 63 that are displayed on display 6 according to Embodiment 1.

In FIG. 6, (a) illustrates physic& condition icon 63 in the case where a total value of sleep period coefficient S and active period coefficient W is 0 or greater and less than 3. Physical condition icon 63 is drawn as a pictogram having a comparatively dam facial expression, and thus physical condition icon 63 indicates that the physical condition of the occupant at the time when the occupant boarded the vehicle is comparatively good.

In FIG. 6, (b) illustrates physical condition icon 63 in the case where a total value of sleep period coefficient S and active period coefficient W is 3 or greater and less than 5. Physical condition icon 63 is drawn as a pictogram having a comparatively fierce facial expression, and thus physical condition icon 63 indicates that the physical condition of the occupant at the time when the occupant boarded the vehicle is worse than the physical condition in the case of (a) in FIG. 6.

In FIG. 6, (c) illustrates physical condition icon 63 in the case where a total value of sleep period coefficient S and active period coefficient W is 5 or greater and less than 10. Physical condition icon 63 is drawn as a pictogram having an extremely fierce facial expression, and thus physical condition icon 63 indicates that the physical condition of the occupant at the time when the occupant boarded the vehicle is worse than the physical condition in the case of (b) in FIG. 6.

Database 16 is implemented using, for example, a semiconductor memory. It is to be noted that database 16 is not particularly limited, and thus can be implemented using a publicly-known means for storing electronic information. Database 16 stores data which are referred to by sleepiness predictor 14 when sleepiness predictor 14 calculates each of route type coefficient L, seep period coefficient S, predicted driving period coefficient Tg, and active period coefficient W, In addition, database 16 stores data which are referred to by sleepiness predictor 14 when sleepiness predictor 14 calculates current sleepiness level N.

### [An Operation]

Hereinafter, an operation that is performed by sleepiness prediction system 100 according to Embodiment 1 is described with reference to FIG. 7, FIG. 7 is a flow chart indicating an example of the operation performed by sleepiness prediction system 100 according to Embodiment 1.

First, when an occupant boards a vehicle and turns on the power of the vehicle, state information obtainer 11 obtains an image of the occupant from camera 2 as state information (S1). In addition, route information obtainer 12 obtains route information including a route which has been set through navigation system 3 and predicted driving period T3 (S2). In addition, lifelog obtainer 13 obtains information including a get-up time and a go-to-bed time of the occupant from wearable device 4 as a part of a lifelog, and obtains information including a boarding time from ECU 5 as a part of the lifelog (S3). It is to be noted that Steps S1 to S3 may be executed in an order different from the above order, or may be executed in parallel.

Next, sleepiness predictor 14 calculates a current sleepiness level of the occupant based on the obtained state information, and predicts sleepiness of the occupant, that is, a sleepiness level (predicted level) of the occupant while the occupant is in the vehicle, based on the current sleepiness level and the obtained route information and lifelog (S4). Notifier 15 then notifies the occupant of the predicted level by displaying the predicted level predicted by sleepiness predictor 14 onto display 6 (S5). In Step S5, physical condition information is notified to the occupant in addition to the predicted level.

Until predetermined period Td elapses after the notification, nothing is particularly executed (S6: No). On the other hand, when predetermined time Td elapses (S6: Yes), sleepiness predictor 14 re-obtains a current sleepiness level, based on state information obtained by state information obtainer 11 (S7). Sleepiness predictor 14 then compares the re-calculated current sleepiness level and the predicted level. When the result of the comparison indicates a match between the current sleepiness level and the predicted level (S8: Yes), sleepiness predictor 14 does not execute any difference correction process. On the other hand, when the result of the comparison indicates a mismatch between the current sleepiness level and the predicted level (S8: Yes), sleepiness predictor 14 executes a difference correction process to correct (update) the predicted level.

Until the vehicle arrives at the destination which has been set (S10: No) after the correction, Steps S6 to S9 described above are repeated. When the vehicle arrives at the destination which has been set (S10: Yes), the operation by sleepiness prediction system 100 ends.

### Advantageous Effects

As described above, sleepiness prediction system 100 according to Embodiment 1 includes lifelog obtainer 13, route information obtainer 12, and sleepiness predictor 14. Lifelog obtainer 13 obtains a lifelog including at least a get-up time of the occupant of the vehicle and a boarding time at which the occupant boards a vehicle. Route information obtainer 12 obtains route information regarding a route to a destination of the vehicle. Sleepiness predictor 14 predicts the sleepiness level of the occupant while the occupant is in the vehicle, based on the lifelog and the route information.

With this, the sleepiness level of the occupant is predicted based on the route to the destination of the vehicle which can affect the sleepiness of the occupant and the get-up time and the boarding time of the occupant. This provides an advantageous effect of being able to increase accuracy in prediction of the sleepiness of the occupant of the vehicle.

In particular, since the sleepiness level of the occupant is predicted based on the get-up time and the boarding time of the occupant, in other words, in consideration of active period T2 from when the occupant gets up to when the occupant boards the vehicle, it is expected that the accuracy in prediction of the sleepiness of the occupant further increases compared with the case in which the sleepiness level of the occupant is predicted without considering active period 12. For example, even when the sleepiness level at the time when the occupant boards the vehicle is the same, the sleepiness level may transit differently depending on the length of the active period before the occupant boards the vehicle.

In addition, in sleepiness prediction system 100 according to Embodiment 1, sleepiness predictor 14 calculates active period coefficient W corresponding to active period T2 which is a difference between the get-up time and the boarding time obtained by lifelog obtainer 13. In addition, sleepiness predictor 14 calculates predicted driving period coefficient Tg corresponding to predicted driving period T3 required to drive the route, based on the route information obtained by route information obtainer 12. In addition, sleepiness predictor 14 calculates route type coefficient L corresponding to a type of the route, based on the route information obtained by route information obtainer 12. Sleepiness predictor 14 then predicts the sleepiness level of the occupant while the occupant is in the vehicle, based on active period coefficient W, predicted driving period coefficient Tg, and route type coefficient L.

This provides an advantageous effect of being able to further increase the accuracy in prediction of the sleepiness level of the occupant while the occupant is in the vehicle in consideration of active period 12 of the occupant, predicted driving period T3 of the vehicle, and the type of the route.

In addition, in sleepiness prediction system 100 according to Embodiment 1, lifelog obtainer 13 further obtains a go-to-bed time of the occupant as a part of the lifelog.

This provides an advantageous effect of being able to further increase the accuracy in prediction of the sleepiness level of the occupant while the occupant is in the vehicle in consideration of the go-to-bed time of the occupant.

In addition, in sleepiness prediction system 100 according to Embodiment 1, sleepiness predictor 14 calculates sleep period coefficient S corresponding to sleep period T1 which is a difference between the go-to-bed time and the get-up time obtained by lifelog obtainer 13. Sleepiness predictor 14 then predicts the sleepiness level of the occupant while the occupant is in the vehicle, further based on sleep period coefficient S.

This provides an advantageous effect of being able to further increase the accuracy in prediction of the sleepiness level of the occupant while the occupant is in the vehicle in consideration of sleep time T1 of the occupant.

In addition, sleepiness prediction system 100 according to Embodiment 1 further includes state information obtainer 11 which obtains state information regarding a state of the occupant at a time of boarding the vehicle. Sleepiness predictor 14 calculates a current sleepiness level of the occupant based on the state information, and predicts the sleepiness level of the occupant while the occupant is in the vehicle, further based on the current sleepiness level.

This provides an advantageous effect of being able to further increase the accuracy in prediction of the sleepiness level of the occupant while the occupant is in the vehicle in consideration of the current sleepiness level of the occupant.

In addition, in sleepiness prediction system 100 according to Embodiment 1, sleepiness predictor 14 re-calculates a current sleepiness level of the occupant every time predetermined time Td has elapsed from the boarding time or an immediately previous predetermined time Td. When a difference is made between the current sleepiness level re-calculated and the sleepiness level of the occupant while the occupant is in the vehicle, sleepiness predictor 14 then corrects the sleepiness level of the occupant while the occupant is in the vehicle so as to eliminate the difference.

This provides an advantageous effect of being able to further increase the accuracy in prediction of the sleepiness level of the occupant while the occupant is in the vehicle by correcting the sleepiness level of the occupant while the occupant is in the vehicle every time predetermined time Td has elapsed.

In addition, sleepiness prediction system 100 according to Embodiment 1 further includes notifier 15 which notifies the occupant of the sleepiness level of the occupant while the occupant is in the vehicle (the sleepiness level of the occupant has been predicted by sleepiness predictor 14 as described above).

This allows the occupant to know the transition in future sleepiness level, which provides an advantageous effect that it is easy for the occupant to take an action for increasing safety in driving the vehicle, for example, by taking a rest.

In addition, in sleepiness prediction system 100 according to Embodiment 1, lifelog obtainer 13 further obtains a go-to-bed time of the occupant as a part of the lifelog. Notifier 15 further notifies the occupant of physical condition information of the occupant based on active period T2 and sleepiness period T1. Active period T2 is a difference between the get-up time and the boarding time obtained by lifelog obtainer 13, and sleep period T1 is a difference between the go-to-bed time and the get-up time obtained by lifelog obtainer 13.

This allows the occupant to know his/her physical condition at the time when the occupant boards the vehicle, which provides an advantageous effect that it is easy for the occupant to know the correlation between the transition in future sleepiness level and his/her physical condition.

In addition, in sleepiness prediction system 100 according to Embodiment 1, state information obtainer 11 obtains, as the state information, an image of the occupant from camera 2 which is mounted on the vehicle and captures an image of the occupant.

With this, it is possible to obtain images of parts having correlation with the sleepiness of the occupant. Examples of the parts include eyelids of the occupant. This provides an advantageous effect that it becomes easy to obtain the current sleepiness level of the occupant.

In addition, in sleepiness prediction system 100 according to Embodiment 1, lifelog obtainer 13 obtains at least a part of the lifelog from wearable device 4 worn by the occupant.

With this, the occupant wears wearable device 4 daily, which provides an advantageous effect that it is easy to increase accuracy in obtainment of the lifelog.

In addition, in sleepiness prediction system 100 according to Embodiment 1, route information obtainer 12 obtains the route information from navigation system 3 mounted on the vehicle.

This provides an advantageous effect that it is easy to obtain the route information compared with the case in which the route information is obtained from the navigation system mounted on an information terminal such as a smartphone.

A sleepiness prediction method according to Embodiment 1 includes: obtaining a lifelog including at least a get-up time of an occupant of a vehicle and a boarding time at which the occupant boards the vehicle (Step S3); obtaining route information regarding a route to a destination of the vehicle (Step S2); and predicting a sleepiness level of the occupant while the occupant is in the vehicle, based on the lifelog and the route information (Step S4).

With this, the sleepiness level of the occupant is predicted based on the route to the destination of the vehicle which can affect the sleepiness of the occupant and the get-up time and the boarding time of the occupant. This provides an advantageous effect of being able to increase accuracy in prediction of the sleepiness of the occupant of the vehicle.

In particular, since the sleepiness level of the occupant is predicted based on the get-up time and the boarding time of the occupant, in other words, in consideration of active period T2 from when the occupant gets up to when the occupant boards the vehicle, it is expected that the accuracy in prediction of the sleepiness of the occupant further increases compared with the case in which the sleepiness level of the occupant is predicted without considering active period T2. For example, even when the sleepiness level at the time when the occupant boards the vehicle is the same, the sleepiness level may transit differently depending on the length of the active period before the occupant boards the vehicle.

### Embodiment 2

### [A Configuration]

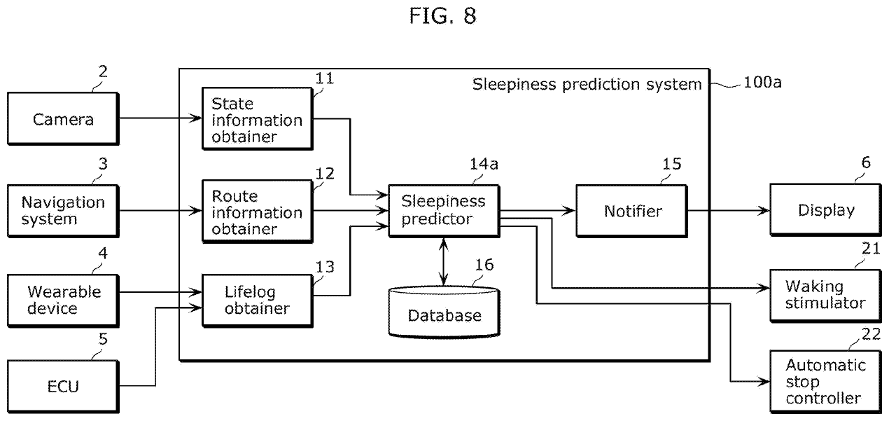

FIG. 8 is a block diagram illustrating an outline of a sleepiness prediction system according to Embodiment 2, Sleepiness prediction system 100a according to Embodiment 2 differs from sleepiness prediction system 100 according to Embodiment 1 mainly in the point that sleepiness predictor 14a is electrically connected to waking stimulator 21 and automatic stop controller 22. Accordingly, the same constituent elements in sleepiness prediction system 100a as in sleepiness prediction system 100 are assigned with the same numerical signs, and detailed descriptions thereof are omitted and differences are described in detail. It is to be noted that occupants comprise not only a driver but also one or more passengers.

Sleepiness predictor 14a is configured similarly to sleepiness predictor 14 according to Embodiment 1, and is electrically connected to waking stimulator 21, Sleepiness predictor 14a is further configured to output, to waking stimulator 21, a driver waking stimulus signal and at least one passenger waking stimulus signal.

Waking stimulator 21 includes, for example, a vibrator (not illustrated) embedded in a driver's sheet in order to wake the driver, Waking stimulator 21 further includes, for example, vibrators (not illustrated) embedded in an assistant driver's sheet and rear sheets in order to wake the passengers. When receiving the driver waking stimulus signal, waking stimulator 21 causes the vibrator in the driver's sheet to operate so as to wake the driver. Likewise, when receiving passenger waking stimulus signals, waking stimulator 21 causes the vibrators in the assistant driver's sheet and rear sheets to operate so as to wake the passengers.

In addition, sleepiness predictor 14a is electrically connected to automatic stop controller 22 and is further configured to output an automatic stop signal for causing a vehicle to stop at a road shoulder.

Automatic stop controller 22 is mounted on the vehicle. When receiving an automatic stop signal, automatic stop controller 22 executes control for causing the vehicle to automatically stop at a road shoulder.

It is to be noted that wearable device 4 according to Embodiment 2 stores, as a part of a lifelog, driver's license holder information of an owner (who can be a driver or a passenger) of wearable device 4. The driver's license holder information is stored in wearable device 4 by means of the owner registering the holding of the driver's license in wearable device 4 in advance. Accordingly, lifelog obtainer 13 obtains the registered driver's license holder information when obtaining another lifelog. The driver's license holder information is output from lifelog obtainer 13 to sleepiness predictor 14a.

### [An Operation]

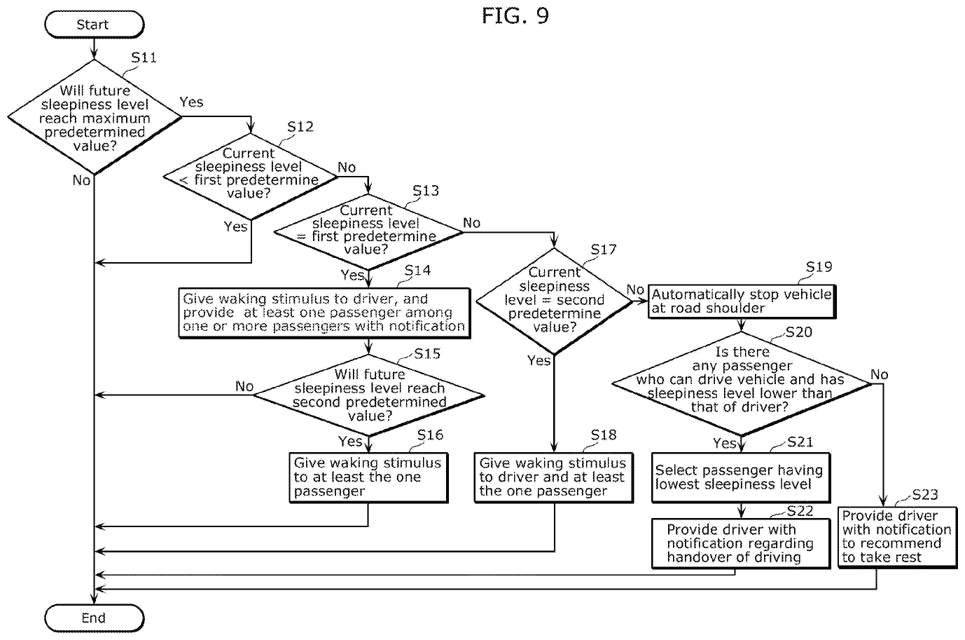

Hereinafter, an operation that is performed by sleepiness prediction system 100a according to Embodiment 2 is described with reference to FIG. 9. FIG. 9 is a flow chart indicating an example of the operation performed by sleepiness prediction system 100a according to Embodiment 2. It is to be noted that descriptions of the same steps in an operation according to Embodiment 2 as steps in the operation according to Embodiment 1 are omitted, and details of steps in the operation which are unique to Embodiment 2 are described.

It is assumed that the sleepiness of the driver and the sleepiness of each of one or more passengers have been respectively predicted based on the operation explained with reference to FIG. 7, on the premise that the operation by sleepiness prediction system 100a illustrated in FIG. 9 is executed.

It is to be noted that the sleepiness of each of the one or more passengers is predicted using the same method as the method for predicting the sleepiness of the driver basically according to the expression (1). At this time, camera 2 is disposed inside the vehicle so that the one or more passengers can also be imaged. Since the one or more passengers do not perform driving, it is assumed that route type coefficient L is “5” indicating a simplest route type and is constant regardless of elapsed time t. In addition, a difference correction process may be performed based on the expressions (2) to (4) also for the prediction of the sleepiness of each of the one or more passengers.

In addition, sleepiness predictor 14a causes, via notifier 15, display 6 to display the sleepiness levels of the driver and each of the one or more passengers calculated as described above. At that time, for example, in graph 61 illustrated in FIG. 5 on display 6, a graph indicating the sleepiness level of the driver and a graph indicating the sleepiness level of each of the one or more passengers are displayed simultaneously. It is to be noted that, when the vehicle incudes a plurality of displays 6, the graph may be displayed on each of displays 6.

Hereinafter, an operation that is performed by sleepiness prediction system 100a illustrated in FIG. 9 is explained. First, sleepiness predictor 14a determines whether a future sleepiness level of the driver (that is, a predicted level of the driver) will reach a maximum predetermined value (S11). Here, the maximum predetermined value is “Level 5” which is a level in the case where it is estimated that the occupant feels extremely sleepy. If the future sleepiness level of the driver will not reach the maximum predetermined value (S11: No), sleepiness predictor 14a ends control in FIG. 9 because sleepiness predictor 14a has predicted that the sleepiness level of the driver will not reach the maximum predetermined value by the time at which the vehicle arrives at a destination.

On the other hand, when a future sleepiness level of the driver will reach the maximum predetermined value (S11: Yes), sleepiness predictor 14a determines whether the current sleepiness level of the driver has reached a first predetermined value (S12). The first predetermined value is, for example, “Level 3” which is a level in the case where it is estimated that the occupant feels sleepy. If the current sleepiness level of the driver has not reached the first predetermined value (S12: Yes), sleepiness predictor 14a ends the control in FIG. 9, On the other hand, the current sleepiness level of the driver has reached or exceeded the first predetermined value (S12: No), sleepiness predictor 14a executes Step S13 and the following steps.

When the current sleepiness level of the driver has reached the first predetermined value (S13: Yes), it is predicted that the driver feels sleepy, Thus, sleepiness predictor 14a outputs, to waking stimulator 21, a driver waking stimulus signal instructing giving of a waking stimulus to the driver. In this way, waking stimulator 21 gives the waking stimulus to the driver. Furthermore, sleepiness predictor 14a notifies, via notifier 15, at least one passenger among the one or more passengers of the fact that the waking stimulus is being given to the driver (S14). It is to be noted that, specifically, sleepiness predictor 14a notifies the one or more passengers of the fact that the waking stimulus is being given to the driver by causing, via notifier 15, display 6 to display information indicating the fact.

Next, sleepiness predictor 14a determines whether a future sleepiness level of the driver will reach a second predetermined value higher than the first predetermined value (S15). Here, the second predetermined value is, for example, “Level 4” which is a level in the case where it is estimated that the occupant feels very sleepy, and which is higher than “Level 3” of the first predetermined value. If it is determined that the future sleepiness level of the driver will not reach the second predetermined value (S15: No), sleepiness predictor 14a ends the control in FIG. 9.

On the other hand, when the future sleepiness level of the driver will reach the second predetermined value (S15: Yes), sleepiness predictor 14a outputs, to waking stimulator 21, a passenger waking stimulus signal instructing giving of a waking stimulus to at least the one passenger (516). In this way, waking stimulator 21 gives the waking stimulus also to the passenger. The reason why the waking stimulus is also given to the passenger is to notify the passenger of taking over of the driving in advance (place the passenger into a state in which the passenger can take over the driving anytime). Thus, it is to be noted that a waking stimulus weaker than the waking stimulus to the driver in Step S14 may be given to the passenger. Sleepiness predictor 14a then ends the control in FIG. 9.

Next, when the current sleepiness level of the driver has exceeded the first predetermined value (S13: No), sleepiness predictor 14a determines whether the current sleepiness level of the driver has reached or exceeded the second predetermined value (“Level 4”) lower than the maximum predetermined value (“Level 5”) (S17). When the current sleepiness level of the driver has reached the second predetermined value (S17: Yes), sleepiness predictor 14a outputs both the driver waking stimulus signal and the at least one passenger waking stimulus signal to waking stimulator 21 (S18). In this way, waking stimulator 21 gives the waking stimuli to both the driver and the passenger.

On the other hand, when the current sleepiness level of the driver has exceeded the second predetermined value (“Level 4”) (S17: No), sleepiness predictor 14a determines that it is difficult for the current driver to continue driving any more, and outputs an automatic stop signal for causing the vehicle to automatically stop at a road shoulder to automatic stop controller 22 (S19). In response to this, automatic stop controller 22 causes the vehicle to automatically stop at the road shoulder. In this way, the driver can safely hand over driving to the passenger selected by sleepiness predictor 14a who is notified in Step S22 to be described later. It is to be noted that the vehicle does not include automatic stop controller 22, there is no need to perform automatic parking at a road shoulder.

Next, sleepiness predictor 14a determines whether there is any passenger whose sleepiness level is lower than that of the driver and who holds a driver's license (S20). It is to be noted that sleepiness predictor 14a obtains the driver's license holder information, which is a part of a lifelog, of each of the one or more passengers, and determines who holds a driver's license among the one or more passengers. If there are passengers each having a sleepiness level lower than that of the driver and holding a driver's license (S20: Yes), sleepiness predictor 14a selects one of the passengers whose sleepiness level is the lowest among the passengers who hold a driver's license (S21). It is to be noted that, when there is only one passenger who holds a driver's license, sleepiness predictor 14a selects the passenger. Sleepiness predictor 14a then provides the driver with a notification regarding handover of driving to the selected appropriate passenger via notifier 15 (S22). Specifically, for example, sleepiness predictor 14a outputs the name of the selected passenger to notifier 15, and notifier 15 causes display 6 to display the name of the passenger. Through such an operation, sleepiness predictor 14a urges the driver to hand over driving to the selected passenger. Sleepiness predictor 14a then ends the control in FIG. 9.

On the other hand, if there is no passenger whose sleepiness level is lower than that of the driver and who holds a driver's license (S20: No), sleepiness predictor 14a provides the driver with a notification to recommend to take a rest via notifier 15 (S23). Specifically, sleepiness predictor 14a outputs, to notifier 15, a signal for causing display 6 to display a message that urges the driver to take a rest. In this way, notifier 15 causes display 6 to display the message that urges the driver to take a rest. Sleepiness predictor 14a then ends the control in FIG. 9. It is to be noted that a case in which there is no passenger whose sleepiness level is lower than that of the driver and who holds a driver's license is either a case in which the sleepiness level of each of the one or more passengers is higher than the current sleepiness level (second predetermined value) of the driver, or a case in which each of the one or more passengers does not hold a driver's license.

### Advantageous Effects

As described above, in sleepiness prediction system 100a according to Embodiment 2: the occupant comprises a driver and one or more passengers; and sleepiness predictor 14a predicts each of a sleepiness level of the driver and a sleepiness level of each of the one or more passengers, and notifies, via notifier 15, each of the driver and the one or more passengers of the sleepiness level of the driver and the sleepiness level of the passenger.

This provides an advantageous effect that also the passenger can know whether the driver will be able to be awake until the vehicle arrives at the destination. This also provides an advantageous effect that the passenger can know the point of time at which switching of drivers will be necessary when it is predicted that the driver will not be able to be awake.

Sleepiness prediction system 100a according to Embodiment 2 further includes waking stimulator 21 which gives a waking stimulus to the occupant. When (i) sleepiness predictor 14a determines that a future sleepiness level of the driver will reach a maximum predetermined value and (ii) the current sleepiness level of the driver reaches a first predetermined value, sleepiness predictor 14a outputs, to waking stimulator 21, a driver waking stimulus signal which instructs giving of a waking stimulus to the driver, and notifies, via notifier 15, at least one of the one or more passengers that the waking stimulus is being given to the driver.

This provides advantageous effects of being able to wake the driver at a stage of a level (first predetermined value) at which the driver feels sleepy, and to notify in advance the passenger of the possibility of taking over of the driving.

In addition, in sleepiness prediction system 100a according to Embodiment 2, when sleepiness predictor 14a determines that the future sleepiness level of the driver will reach a second predetermined value higher than the first predetermined value, sleepiness predictor 14a outputs, to waking stimulator 21, a passenger waking stimulus signal which instructs giving of a waking stimulus to the at least one of the one or more passengers.

This provides an advantageous effect of being able to give the waking stimulus to the passenger to provide the passenger with pre-notification regarding the taking over of the driving.

In addition, in sleepiness prediction system 100a according to Embodiment 2, when sleepiness predictor 14a determines that (i) the future sleepiness level of the driver will reach a maximum predetermined value and (ii) the current sleepiness level of the driver exceeds a second predetermined value lower than the maximum predetermined value, sleepiness predictor 14a notifies, via notifier 15, the driver and a passenger having a lower sleepiness level than the current sleepiness level of the driver among the one or more passengers of switching drivers from the driver to the passenger having the lower sleepiness level.

With this, switching to the passenger having a sleepiness level lower than that of the driver is urged, which provides an advantageous effect of being able to continue safety driving to the destination.

In addition, in sleepiness prediction system 100a according to Embodiment 2, when the one or more passengers comprise a plurality of passengers, sleepiness predictor 14a notifies, via notifier 15, at least a passenger having a lowest sleepiness level among the plurality of passengers of switching drivers from the driver to the passenger having the lowest sleepiness level.

This provides an advantageous effect of increasing the likelihood of safety driving to the destination by means of the passenger having the lowest sleepiness taking over the driving among all the occupants.

In addition, sleepiness prediction system 100a according to Embodiment 2 further includes automatic stop controller 22 which executes control for causing the vehicle to automatically stop. When sleepiness predictor 14a notifies, via notifier 15, the driver and the passenger of switching of drivers from the driver to the passenger, sleepiness predictor 14a outputs, to automatic stop controller 22, an automatic stop signal for causing the vehicle to automatically stop at a road shoulder.

This provides an advantageous effect of enabling safety stop of the vehicle and switching to the passenger.

In addition, in sleepiness prediction system 100a according to Embodiment 2: lifelog obtainer 13 further obtains, as a part of the lifelog, driver's license holder information of a passenger who holds a driver's license among the one or more passengers; and sleepiness predictor 14a notifies, via notifier 15, at least the passenger who holds the driver's license of switching drivers from the driver to the passenger who holds the driver's license.

This provides an advantageous effect of enabling switching to the more appropriate passenger.

In addition, sleepiness prediction system 100a according to Embodiment 2 further includes notifier 15 which notifies the occupant of the sleepiness level of the occupant while the occupant is in the vehicle (the sleepiness level of the occupant has been predicted by sleepiness predictor 14a as described above). The occupant comprises a driver and one or more passengers. Sleepiness predictor 14a: predicts each of a sleepiness level of the driver and a sleepiness level of each of the one or more passengers; predicts the sleepiness level of the passenger using a coefficient in a case where the route is simplest as route type coefficient L, when predicting the sleepiness level of the passenger; and notifies, via notifier 15, each of the driver and the passenger of the predicted sleepiness level of the driver and the predicted sleepiness level of the passenger.

This provides an advantageous effect that also the passenger can know whether the driver will be able to be awake until the vehicle arrives at the destination. This also provides an advantageous effect that the passenger can know the point of time at which switching of drivers will be necessary when it is predicted that the driver will not be able to be awake. Furthermore, since the sleepiness level of the passenger is predicted using route type coefficient L in the case where the route is simplest, an advantageous effect of being able to predict the sleepiness level of the passenger who does not drive the vehicle more accurately is provided.

### Variations

Although the sleepiness prediction system according to the present disclosure has been described based on each of the above embodiments, the present disclosure is not limited to the embodiments. The present disclosure may cover and encompass embodiments that a person skilled in the art may arrive at by adding various kinds of modifications to any of the above embodiments within the scope of the present disclosure.

Although state information obtainer 11 obtains state information from the image of the occupant captured by camera 2 mounted on the vehicle, the way of obtaining state information is not limited thereto. For example, when a mobile terminal such as a smartphone or wearable device 4 of the occupant has a function of measuring a heart rate of the occupant, state information obtainer 11 may obtain, as state information, information including the heart rate of the occupant from such a device by performing wireless communication with the device. The heart rate is information which is likely to express a tendency according to a sleepiness of the occupant as with the positions of the eyelids, etc., of the occupant.

Although route information obtainer 12 obtains route information from navigation system 3 mounted on the vehicle in each of the embodiments, the way of obtaining route information is not limited thereto. For example, when a mobile terminal such as a smartphone of the occupant has one or more functions of a navigation system different from the navigation system mounted on the vehicle, route information obtainer 12 may obtain route information from the mobile terminal by performing wireless communication with the mobile terminal.

Although lifelog obtainer 13 obtains information including the get-up time and the go-to-bed time included in the lifelog from wearable device 4 worn by the occupant in each of the embodiments, the way of obtaining the information is not limited thereto. For example, when a mobile terminal such as a smartphone of the occupant has a function of measuring numerical data indicating an activity of the occupant, lifelog obtainer 13 may obtain information including a get-up time and a go-to-bed time from the mobile device by performing wireless communication with the mobile terminal. In addition, for example, when a sensor for monitoring sleep is disposed beside the bed of the occupant, the mobile terminal may obtain information including a get-up time and a go-to-bed time from the sensor through wireless communication with the sensor. In this case, lifelog obtainer 13 is capable of indirectly obtaining the information including the get-up time and the go-to-bed time from the sensor by performing wireless communication with the mobile terminal. Alternatively, lifelog obtainer 13 may be configured to obtain a part of a lifelog from a stationary-type device that is for example a bed capable of obtaining the part of the lifelog. Furthermore, lifelog obtainer 13 may be configured to obtain parts of a lifelog from both wearable device 4 and either the sensor or the stationary-type device.

In each of the above embodiments, a corresponding one of sleepiness prediction systems 100 and 100a may be implemented in the form of, for example, an in-vehicle infotainment (WI) system which is an in-vehicle information communication system using a car navigation system, a display, and an audio device.

Although each of sleepiness predictors 14 and 14a determines whether to perform a process for correcting a difference in predicted level every time predetermined time Td has elapsed from the boarding time or an immediately previous predetermined time Td in the above embodiments, the way of difference correction is not limited thereto. For example, each of sleepiness predictors 14 and 14a may be configured to predict sleepiness of the occupant while the occupant is in the vehicle only at the time of boarding the vehicle without making such a determination every time predetermined time Td has elapsed.

In each of the above embodiments, it is only necessary that a corresponding one of sleepiness predictors 14 and 14a be configured to predict sleepiness of the driver while the driver is in the vehicle based on at least the get-up time, the boarding time, and the route information which are included in the lifelog. Accordingly, each of sleepiness predictors 14 and 14a does not always need to refer to the go-to-bed time included in the lifelog and the state information when predicting the sleepiness level of the occupant while the occupant is in the vehicle. In this case, state information obtainer 11 is unnecessary, and lifelog obtainer 13 does not always need to obtain the go-to-bed time.

In addition, although each of sleepiness predictors 14 and 14a determines each of the sleepiness levels to be a sleepiness level at one of the 4 stages in the above embodiments, the way of determining the sleepiness level is not limited thereto. For example, each of sleepiness predictors 14 and 14a may determine each of the sleepiness levels to be a sleepiness level at one of stages the number of which is greater than 5 or smaller than 5.

In each of the embodiments, although notifier 15 visually notifies the occupant of the predicted level by causing display 6 to display information including the predicted level, the way of notifying the information is not limited thereto. For example, notifier 15 may auditorily notify the occupant of the predicted level by outputting, in form of a sound, information including the predicted level through a speaker mounted on the vehicle. In addition, notifier 15 may visually and auditorily notify the occupant of the predicted level in combination with the display by display 6 and the sound output through the speaker.

In addition, although notifier 15 further notifies the occupant of the physical condition information in addition to the predicted level in each of the above embodiments, information to be notified is not limited thereto. For example, notifier 15 may notify the occupant of the predicted level only.

In addition, although notifier 15 notifies the occupant of the information that directly indicates the predicted level predicted by sleepiness predictor 14 or 14a in each of the above embodiments, the way of notifying the information is not limited thereto. For example, notifier 15 may notify the occupant of advice based on the predicted level. Examples of such advance includes “[w]hy don't you take a rest at an early timing because you may become sleepy after a predetermined time.” In this mode, it can be said that notifier 15 notifies the occupant of information indirectly indicating a predicted level.

In addition, although each of sleepiness prediction systems 100 and 100a in the above embodiments is implemented as an in-vehicle device, the way of implementing sleepiness prediction systems 100 and 100a is not limited thereto. For example, each of sleepiness prediction systems 100 and 100a may be mounted on a server device outside the vehicle, and may be configured to predict sleepiness of an occupant by performing wireless communication with the server device. Alternatively, each of sleepiness prediction systems 100 and 100a may have a complex configuration composed of, for example, some part mounted on a vehicle and the remaining part mounted on a server device.

Each of elements included in each of sleepiness prediction systems 100 and 100a according to the above embodiments is typically implemented as a large-scale integration (LSI) which is an integrated circuit. The elements may be made as separate individual chips, or as a single chip to include a part or all thereof.

The means for circuit integration is not limited to an LSI, and implementation with a dedicated circuit or a general-purpose processor is also available. It is also possible to use a field programmable gate array (FPGA) that is programmable after the LSI is manufactured, and a reconfigurable processor in which connections and settings of circuit cells within the LSI are reconfigurable.

It is to be noted that, in the above embodiments, each of the elements may be configured with dedicated hardware, or may be implemented by executing a software program suitable for the element. Each of the elements may be implemented by means of a program executer such as a central processing unit (CPU) or a processor reading out a software program recorded on a recording medium such as a hard disc or a semiconductor memory and executing the software program.

In addition, all the numerals used above are intended to indicate examples for specifically explaining the present disclosure, and thus each of the embodiments of the present disclosure is not limited to the numerals indicated as the examples.

In addition, the partitioning into the functional blocks in each of block diagrams is one example. It is also possible to implement a plurality of functional blocks as one functional block, partition one functional block into a plurality of blocks, or move a part of a function to another block. In addition, the functions of the plurality of functional blocks having similar functions may be processed by a single hardware or software product in parallel or in time division.

In addition, the order of executing the steps in each of the flow charts is indicated as an example for specifically explaining the present disclosure, and thus another order is also possible. In addition, part of the steps may be executed at the same time (in parallel) with another one of the steps.

The present disclosure covers and encompasses other embodiments that a person skilled in the art may arrive at by adding various kinds of modifications to the above embodiments or by optionally combining some of the elements and functions in any of the embodiments within the scope of the present disclosure.

While various embodiments have been described herein above, it is to be appreciated that various changes in form and detail may be made without departing from the spirit and scope of the present disclosure as presently or hereafter claimed.

### FURTHER INFORMATION ABOUT TECHNICAL BACKGROUND TO THIS APPLICATION

The disclosures of the following Japanese Patent Applications including specification, drawings and claims are incorporated herein by reference in their entirety: Japanese Patent Application No. 2021-141314 filed on Aug. 31, 2021, and priority of Japanese Patent Application No. 2022-066876 filed on Apr. 14, 2022.

### INDUSTRIAL APPLICABILITY

The present disclosure is applicable to systems for supporting driving of vehicles in moving bodies such as vehicles, etc.

1. A sleepiness prediction system comprising:
a lifelog obtainer which obtains a lifelog including at least a get-up time of an occupant of a vehicle and a boarding time at which the occupant boards the vehicle;
a route information obtainer which obtains route information regarding a route to a destination of the vehicle; and
a sleepiness predictor which predicts a sleepiness level of the occupant while the occupant is in the vehicle, based on the lifelog and the route information.

2. The sleepiness prediction system according to claim 1,
wherein the sleepiness predictor:
calculates an active period coefficient corresponding to an active period which is a difference between the get-up time and the boarding time obtained by the lifelog obtainer;
calculates a predicted driving period coefficient corresponding to a predicted driving period required to drive the route, based on the route information obtained by the route information obtainer;
calculates a route type coefficient corresponding to a type of the route, based on the route information obtained by the route information obtainer; and
predicts the sleepiness level of the occupant while the occupant is in the vehicle, based on the active period coefficient, the predicted driving period coefficient, and the route type coefficient.

3. The sleepiness prediction system according to claim 1,
wherein the lifelog obtainer further obtains a go-to-bed time of the occupant as a part of the lifelog.

4. The sleepiness prediction system according to claim 3,
wherein the sleepiness predictor:
calculates a sleep period coefficient corresponding to a sleep period which is a difference between the go-to-bed time and the get-up time obtained by the lifelog obtainer; and
predicts the sleepiness level of the occupant while the occupant is in the vehicle, further based on the sleep period coefficient.

5. The sleepiness prediction system according to claim 1, further comprising:
a state information obtainer which obtains state information regarding a state of the occupant at a time of boarding the vehicle,
wherein the sleepiness predictor calculates a current sleepiness level of the occupant based on the state information, and predicts the sleepiness level of the occupant while the occupant is in the vehicle, further based on the current sleepiness level.

6. The sleepiness prediction system according to claim 5,
wherein the sleepiness predictor:
re-calculates a current sleepiness level of the occupant every time a predetermined time has elapsed from the boarding time or an immediately previous predetermined time; and
when a difference is made between the current sleepiness level re-calculated and the sleepiness level of the occupant while the occupant is in the vehicle, corrects the sleepiness level of the occupant while the occupant is in the vehicle so as to eliminate the difference.

7. The sleepiness prediction system according to claim 1, further comprising:
a notifier which notifies the occupant of the sleepiness level of the occupant while the occupant is in the vehicle, the sleepiness level of the occupant being predicted by the sleepiness predictor.

8. The sleepiness prediction system according to claim 7,
wherein the lifelog obtainer further obtains a go-to-bed time of the occupant as a part of the lifelog; and
the notifier further notifies the occupant of physical condition information of the occupant based on an active period and a sleep period, the active period being a difference between the get-up time and the boarding time obtained by the lifelog obtainer, the sleep period being a difference between the go-to-bed time and the get-up time obtained by the lifelog obtainer.

9. The sleepiness prediction system according to claim 5,
wherein the state information obtainer obtains, as the state information, an image of the occupant from a camera which is mounted on the vehicle and captures an image of the occupant.

10. The sleepiness prediction system according to claim 1,
wherein the lifelog obtainer obtains at least a part of the lifelog from a wearable device worn by the occupant.

11. The sleepiness prediction system according to claim 1,
wherein the route information obtainer obtains the route information from a navigation system mounted on the vehicle.

12. The sleepiness prediction system according to claim 7,
wherein the occupant comprises a driver and one or more passengers, and
the sleepiness predictor predicts each of a sleepiness level of the driver and a sleepiness level of each of the one or more passengers, and notifies, via the notifier, each of the driver and the one or more passengers of the sleepiness level of the driver and the sleepiness level of the passenger.

13. The sleepiness prediction system according to claim 12, further comprising:
a waking stimulator which gives a waking stimulus to the occupant,
wherein, when (i) the sleepiness predictor determines that a future sleepiness level of the driver will reach a maximum predetermined value and (ii) the current sleepiness level of the driver reaches a first predetermined value, the sleepiness predictor outputs, to the waking stimulator, a driver waking stimulus signal which instructs giving of a waking stimulus to the driver, and notifies, via the notifier, at least one of the one or more passengers that the waking stimulus is being given to the driver.

14. The sleepiness prediction system according to claim 13,
wherein, when the sleepiness predictor determines that the future sleepiness level of the driver will reach a second predetermined value higher than the first predetermined value, the sleepiness predictor outputs, to the waking stimulator, a passenger waking stimulus signal which instructs giving of a waking stimulus to the at least one of the one or more passengers.

15. The sleepiness prediction system according to claim 12,
wherein, when the sleepiness predictor determines that (i) a future sleepiness level of the driver will reach a maximum predetermined value and (ii) the current sleepiness level of the driver exceeds a second predetermined value lower than the maximum predetermined value, the sleepiness predictor notifies, via the notifier, the driver and a passenger having a lower sleepiness level than the current sleepiness level of the driver among the one or more passengers of switching drivers from the driver to the passenger having the lower sleepiness level.

16. The sleepiness prediction system according to claim 15,
wherein, when the one or more passengers comprise a plurality of passengers, the sleepiness predictor notifies, via the notifier, at least a passenger having a lowest sleepiness level among the plurality of passengers of switching drivers from the driver to the passenger having the lowest sleepiness level.

17. The sleepiness prediction system according to claim 15, further comprising:
an automatic stop controller which executes control for causing the vehicle to automatically stop,
wherein, when the sleepiness predictor notifies, via the notifier, the driver and the passenger of switching of drivers from the driver to the passenger, the sleepiness predictor outputs, to the automatic stop controller, an automatic stop signal for causing the vehicle to automatically stop at a road shoulder.

18. The sleepiness prediction system according to claim 15,
wherein the lifelog obtainer further obtains, as a part of the lifelog, driver's license holder information of a passenger who holds a driver's license among the one or more passengers, and
the sleepiness predictor notifies, via the notifier, at least the passenger who holds the driver's license of switching of drivers from the driver to the passenger who holds the driver's license.

19. The sleepiness prediction system according to claim 2, further comprising:
a notifier which notifies the occupant of the sleepiness level of the occupant while the occupant is in the vehicle, the sleepiness level of the occupant being predicted by the sleepiness predictor,
wherein the occupant comprises a driver and one or more passengers,
the sleepiness predictor:
predicts each of a sleepiness level of the driver and a sleepiness level of each of the one or more passengers;
predicts the sleepiness level of the passenger using a coefficient in a case where the route is simplest as the route type coefficient, when predicting the sleepiness level of the passenger; and
notifies, via the notifier, each of the driver and the passenger of the predicted sleepiness level of the driver and the predicted sleepiness level of the passenger.

20. A sleepiness prediction method comprising:
obtaining a lifelog including at least a get-up time of an occupant of a vehicle and a boarding time at which the occupant boards the vehicle;
obtaining route information regarding a route to a destination of the vehicle; and
predicting a sleepiness level of the occupant while the occupant is in the vehicle, based on the lifelog and the route information.
